(19) United States
(12) Patent Application Publication
(10) Pub. No.: US 2021/0330259 A1
(43) Pub. Date: Oct. 28, 2021

(54) VITAL-MONITORING MASK

(71) Applicant: Vita Innovations, Inc., New Orleans, LA (US)

(72) Inventors: Longsha Liu, New Orleans, LA (US); Kristen V. Ong, North Massapequa, NY (US); Rishi Singhal, Jericho, NY (US); Jason W. Chen, Green Cove Springs, FL (US); Ray Wei, Stony Brook, NY (US)

(21) Appl. No.: 17/243,114

(22) Filed: Apr. 28, 2021

Related U.S. Application Data
(60) Provisional application No. 63/016,799, filed on Apr. 28, 2020.
(60) Provisional application No. 63/116,916, filed on Nov. 22, 2020.

(51) Int. Cl. A61B 5/00; A61B 5/0205; A61B 5/1455; A61B 5/389

(52) U.S. Cl. CPC A61B 5/6803; A61B 5/02055; A61B 5/14552; A61B 5/389; A61B 5/02438; A61B 5/6815; A61B 5/0022; A61B 5/7264; A61B 5/6843; A61B 5/0077

(57) ABSTRACT

The apparatus presented herein may be used for inhibiting or preventing spread of airborne pathogens while providing immediate, continuous and remote patient vital monitoring and diagnosis, without requiring medical staff supervision. In some embodiments, a mask and earpiece is provided that is configured to measure body temperature, heart rate, blood oxygen levels and respiratory rate.

### CROSS-REFERENCE TO RELATED APPLICATIONS

This application claims the benefit of U.S. Provisional Patent Application Nos. 63/016,799, filed on Apr. 28, 2020, and 63/116,916, filed on Nov. 22, 2020, each of which is hereby incorporated by reference in its entirety.

### FIELD OF THE INVENTION

Embodiments of the present disclosure relate to apparatuses and systems for collection and monitoring of patient data, while ensuring patient and physician safety. In particular, the present disclosure is directed towards a removable face mask including an array of interconnected (e.g., IoT) sensors to monitor patient vitals.

### BACKGROUND

A common situation observed in large urban hospitals is patients waiting hours to receive care in the emergency department, not at the fault of the hospital staff, but simply due to the lack of sufficient personnel to respond to overwhelming demand for care. This problem, known as emergency department (ED) overcrowding, is prevalent worldwide with few solutions. As a result, patients may end up waiting anywhere from 4 to 12 hours before receiving medical attention, each second of which can lead to increased walkouts, mortality, & progression of symptoms. With COVID-19, this issue of ED overcrowding has exacerbated with a compounded fear of contracting a respiratory illnesses. Additionally, during severe epidemics, there is a high risk for viral transmission in waiting areas and standard one-use surgical masks are not effective enough to protect patients.

Hospitals are unable to institute continuous monitoring procedures in waiting rooms due to lack of staff and equipment. Existing continuous monitoring technologies measure one or two vitals at a time and may be connected to bulky machines that do not fit into a crowded waiting area. This can have large impacts on a hospital's bottom line as an ED can experience a $600-$800 loss in revenue for every insured walkout. In addition, there have recently been a string of lawsuits filed against hospitals for unmonitored decline of health as patients await care in the waiting room. While most of these cases were settled out of court, they had significant financial and reputational costs to the hospitals.

Accordingly, there is a need for an efficient and economic method and system for optimizing collection and monitoring of patient data by providing a removable face mask that includes an array of interconnected (e.g., IoT) sensors to monitor patient vitals as a triage-assisted medical technology.

### BRIEF SUMMARY

In various embodiments, an apparatus is provided including a respiratory mask having a gasket configured to form a seal against skin around a nose and a mouth of a user. The mask has a breath sensor, at least one opening, and a filter disposed in the at least one opening. The apparatus further includes a sensing device comprising a temperature sensor, a blood oxygen saturation sensor, and a heart rate sensor. The sensing device is configured to attach to an external body part of the user and externally from the mask. The apparatus further includes an electronics housing disposed on the mask. The electronics housing includes a power source and a processor in electrical communication with the breath sensor, the temperature sensor, the blood oxygen saturation sensor, and the heart rate sensor. The electronics housing further includes a computer readable storage medium having program instructions embodied therewith, and the program instructions are executable by the processor to cause the processor to perform a method where breathing rate data is received from the breath sensor, temperature data is received from the temperature sensor, blood oxygen saturation data is received from the blood oxygen saturation sensor, heart rate data is received from the heart rate sensor, and the breathing rate data, temperature data, blood oxygen saturation data, and heart rate data is transmitted to an external device.

In various embodiments, a system is provided including the apparatus described above and the external device. In various embodiments, the external device is a mobile device. In various embodiments, the mobile device is a cell phone. In various embodiments, the external device is a remote server. In various embodiments, the remote server is an electronic medical record (EMR) server.

In various embodiments, a vital-monitoring apparatus is provided including a sensing device comprising a temperature sensor, a blood oxygen saturation sensor, and a heart rate sensor, the sensing device configured to attach to an external body part of a user. The apparatus further includes an electronics housing configured to be removably attached to a respiratory mask. The electronics housing includes a power source and a processor in electrical communication with the breath sensor, the temperature sensor, the blood oxygen saturation sensor, and the heart rate sensor. The electronics housing further includes a computer readable storage medium having program instructions embodied therewith, and the program instructions are executable by the processor to cause the processor to perform a method where breathing rate data is received from the breath sensor, temperature data is received from the temperature sensor, blood oxygen saturation data is received from the blood oxygen saturation sensor, heart rate data is received from the heart rate sensor, and the breathing rate data, temperature data, blood oxygen saturation data, and heart rate data are transmitted to an external device.

In various embodiments, a kit is provided including the apparatus described above and the respiratory mask onto which the electronics housing is configured to removably attach.

In various embodiments, a vital-monitoring device is provided including a sensing device having a temperature sensor, a blood oxygen saturation sensor, and a heart rate sensor. The sensing device is configured to attach to an external body part of a user.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIGS. 1A-1F illustrates a schematic representation of a wearable biosensor mask in accordance with an embodiment of the present disclosure.

FIGS. 2A-2F illustrate an exemplary vital-monitoring mask in accordance with an embodiment of the present disclosure. FIG. 2G illustrates an exemplary earpiece in accordance with an embodiment of the present disclosure. FIG. 2H illustrates an exemplary vital-monitoring mask and earpiece in accordance with an embodiment of the present disclosure.

FIG. 3 illustrates an exemplary vital-monitoring mask in accordance with an embodiment of the present disclosure.

FIG. 4 illustrates an exemplary vital-monitoring mask in accordance with an embodiment of the present disclosure.

FIGS. 5A-5B illustrate a back view (FIG. 5A) and a front view (FIG. 5B) of the vital-measuring gasket mask with a built-in enclosure for insertion of the electronics in accordance with an embodiment of the present disclosure.

FIG. 6 illustrates a protective electronics casing that houses the electronic components in accordance with an embodiment of the present disclosure.

FIGS. 7A-7B illustrate a front view (FIG. 7A) and a back view (FIG. 7B) of a pulse oximeter earpiece component in accordance with an embodiment of the present disclosure.

FIG. 8A illustrates an earpiece clamp that may house the pulse oximeter for clamping on the earlobe in accordance with an embodiment of the present disclosure. FIG. 8B illustrates an expanded view of the earpiece clamp in accordance with an embodiment of the present disclosure.

FIG. 9 illustrates a patient wearing the mask, with zoomed in windows of the pulse oximeter attached to the earlobe, connected via a retractable reel to the gasket mask on the face in accordance with an embodiment of the present disclosure.

FIGS. 10A-10E illustrate schematics of a printed circuit board (PCB) in accordance with an embodiment of the present disclosure.

FIGS. 11A-11B illustrate a 3D model of the PCB in accordance with an embodiment of the present disclosure.

FIG. 12 illustrates a velostat sensor design in accordance with an embodiment of the present disclosure.

FIG. 13 illustrates a schematic representation of an electrical system of a wearable biosensor mask in accordance with an embodiment of the present disclosure.

FIG. 14 depicts an exemplary computing node according to embodiments of the present disclosure.

### DETAILED DESCRIPTION

Reference will now be made in detail to exemplary embodiments of the disclosed subject matter, an example of which is illustrated in the accompanying drawings. The method and corresponding steps of the disclosed subject matter will be described in conjunction with the detailed description of the system.

In various embodiments, an array of interconnected, e.g., internet-of-things (IoT), sensors are incorporated into a respiratory filtration mask such that medical personnel (e.g., doctor, nurse, paramedic, EMT, etc.) may attach the mask to a patient in order to immediately begin monitoring key vitals including, for example, body temperature, heart rate, blood oxygen levels, respiratory rate, etc., as the medical personnel perform other essential tasks. Additionally, the mask itself prevents the transmission of airborne viral particles between patients in proximity to one another. Thus, unlike other solutions which require constant attention of the health care provider to administer and monitor these diagnostic tests, the mask apparatus disclosed herein provides for continuous monitoring and diagnosis without health care provider participation, while also reducing transmission rates in the emergency room or waiting area.

In various embodiments, the apparatus presented herein may be used for inhibiting or preventing spread of airborne pathogens while providing immediate, continuous and remote patient vital monitoring and diagnosis, without requiring medical staff supervision.

In various embodiments, the apparatus of the present disclosure may include a mask and an external sensing device (e.g., an earpiece). In various embodiments, the mask may be made from any suitable known materials, such as, for example, a polymer. In various embodiments, the mask may include a gasket seal for a more secure adhesion to the skin, especially around the nose-bridge region. In various embodiments, the mask can be 3D-printed with polylactic acid (PLA). In various embodiments, the mask may include a disposable filter cartridge (e.g., N95, N100) to allow for reusability while still preventing the transmission of viral particles. In various embodiments, the mask may be made of a material that is washable and reusable. In various embodiments, the mask, including the gasket seal, may be disposable upon use while the electronics are reused.

In various embodiments, the mask may be formed of a polymer resin. In various embodiments, the mask may be formed of a suitable textile (e.g., non-woven fabric). In various embodiments, the mask may contain a sensor(s) for measuring breathing rate of the user. In various embodiments, the breath sensor may be a microphone. In various embodiments, the breath sensor may be a pressure sensor. In various embodiments, the apparatus may include additional components integrated into a sublayer, e.g., on one or both side(s) of the mask. In various embodiments, the additional components may be a printed circuit board (PCB), a Bluetooth module (e.g., to connect to the hospital server), and/or a power source (e.g., a rechargeable lithium battery or disposable battery). In various embodiments, muscle changes may also be measured by EMG sensors.

In various embodiments, the external sensing device (e.g., earpiece) may be integrated with a strap that fastens the mask onto the face of the user. In various embodiments, the earpiece may include an infrared temperature sensor. In various embodiments, the temperature sensor may rest inside a patient's ear, similar to an earbud. In various embodiments, a portion of the earpiece may contain a blood oxygen saturation sensor (e.g., pulse oximeter) configured to measure a blood oxygen saturation of a user. In various embodiments, the earpiece may be fastened on either side of the patient's earlobe. In various embodiments, the temperature sensor may measure body temperature over time. In various embodiments, the pulse oximeter can measure both blood oxygen (SPO2) levels and heart rate over time. In various embodiments, the breathing rate, body temperature, blood oxygen saturation (SPO₂) levels, and/or heart rate may be measured by the apparatus.

In various embodiments, one or more wires may connect the external sensing device to the central PCB on the mask. In various embodiments, one or more wires may be embedded within a neoprene strap that is configured to wrap around the user's head. In various embodiments, the one or more wires may be housed in a protective shroud that is coupled (e.g., stitched) to the strap(s). In various embodiments, the one or more wires may include mating prongs/ports to allow the wires (and/or straps if so desired) to be removed or detached from the sensor(s) to allow for repair and replacement of damaged wires/sensors. In various embodiments, data from the sensors can be recorded at a processor. In various embodiments, the processor may be embedded in the central PCB. In various embodiments, the recorded data may be wirelessly transmitted (e.g., via Bluetooth) to a desktop monitor to display the data. In various embodiments, the recorded data may be wirelessly transmitted to a mobile device (e.g., mobile phone, tablet, laptop, etc.). In various embodiments, the recorded data may be transmitted to a remote server (e.g., an electronic medical record server). In various embodiments, the data may be streamed to another device (e.g., mobile device, server, etc.). In various embodiments, the sensors in the external sensing device may be covered with a polymer covering. In various embodiments, the external sensing device may be sanitized with known antiseptics (e.g., Isopropyl alcohol and/or hibiclens solution) allowing for reusability.

In various embodiments, the apparatus may be configured with a single strap acting as the mechanical housing of the various components described above (e.g., PCB, sensors, etc.) instead of being housed within a mask. In various embodiments, the single strap may be positioned above the nose, akin to a visor design. In various embodiments, the apparatus may only include the infrared sensor and pulse oximeter measuring body temperature, SPO2 levels, and pulse rate. In various embodiments, the single strap configuration may house the majority of non-sensor components in a region between the inner and outer bands.

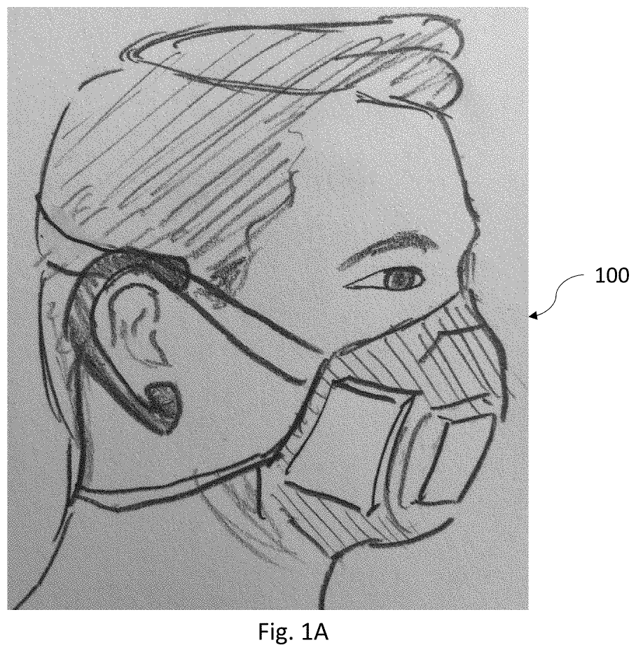

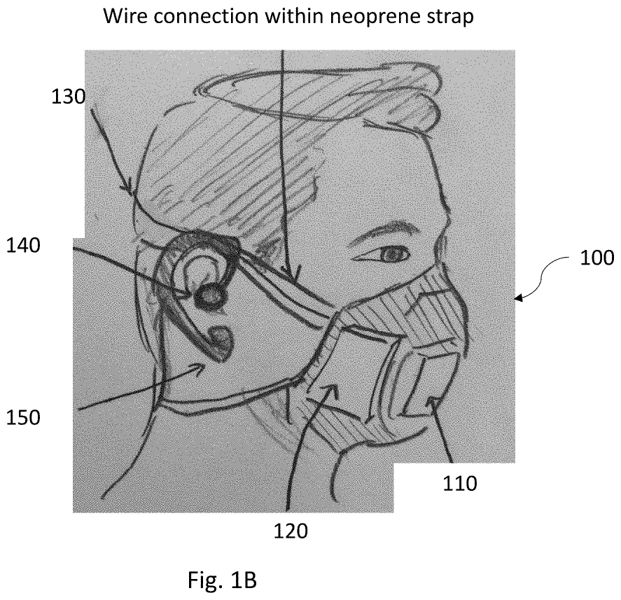

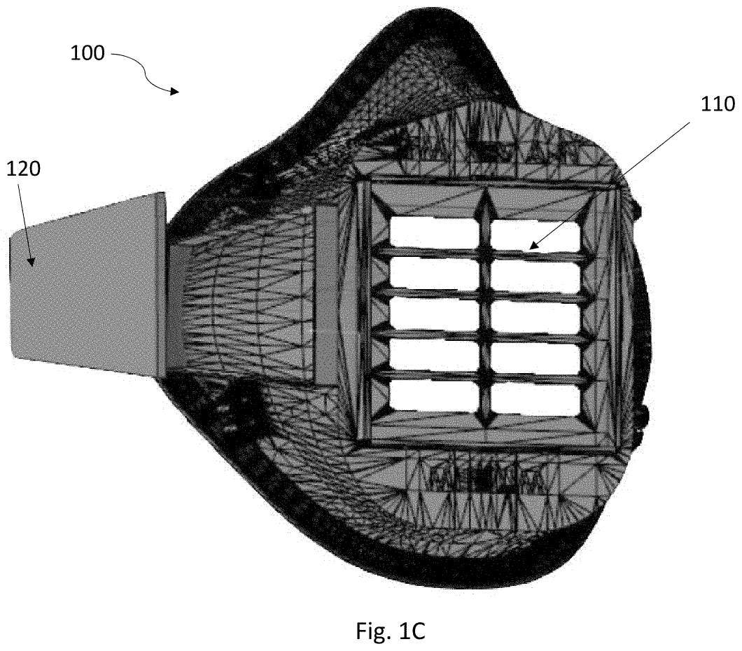

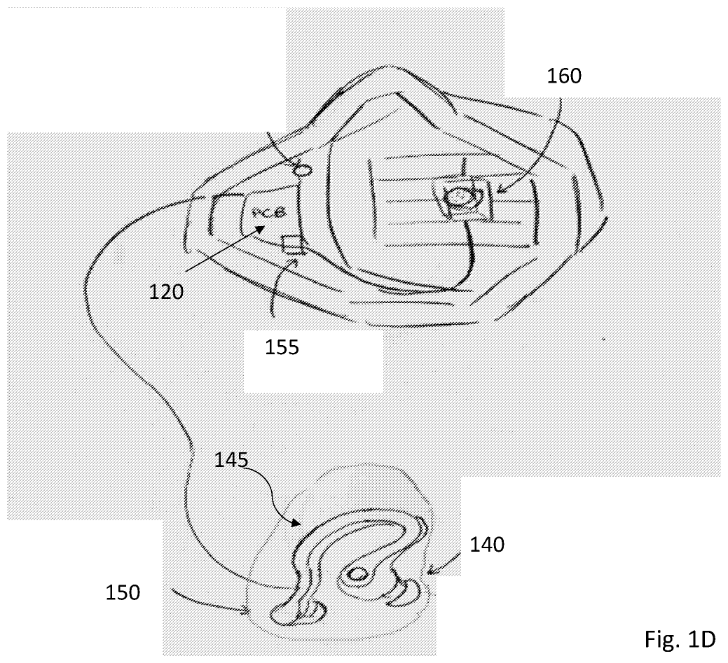

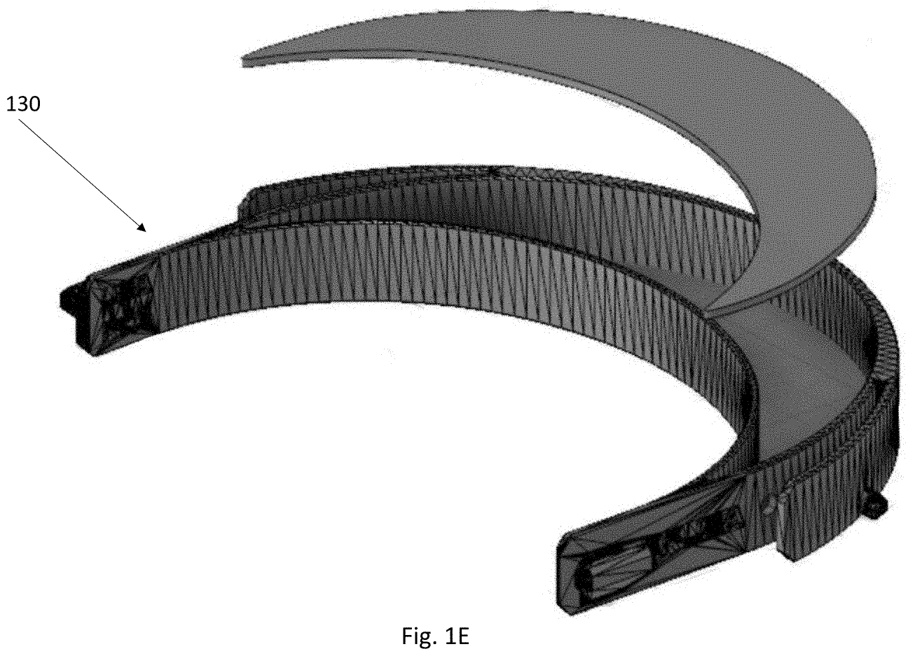

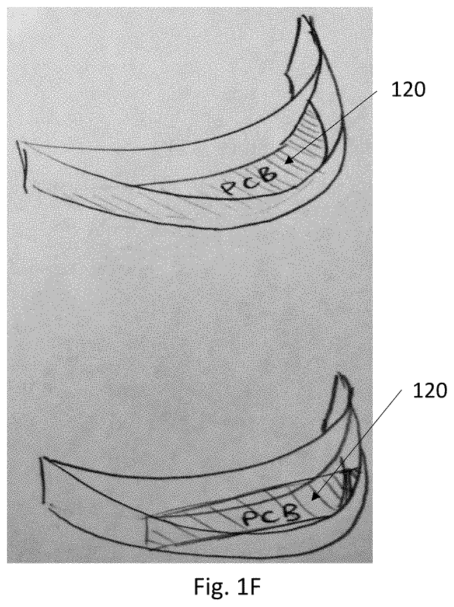

For purpose of explanation and illustration, and not limitation, an exemplary embodiment of the apparatus in accordance with the disclosed subject matter is shown in FIGS. 1A-1D and is designated generally by reference character 100. Similar reference numerals (differentiated by the leading numeral) may be provided among the various views and Figures presented herein to denote functionally corresponding, but not necessarily identical structures.

In various embodiments, as shown in FIGS. 1A-1D, the apparatus 100 may include a facial mask sized and shaped to cover a patient's nose and mouth. In various embodiments, the apparatus can include a filter 110 disposed at the front of the mask, and a printed circuit board 120 disposed on one or both side(s) of the mask. In various embodiments, one or more straps 130 (e.g., one or more neoprene straps) can be coupled to the top and bottom of the mask and sized to extend behind the user's head. In various embodiments, the straps are adjustable in length, and/or removable and replaceable. In various embodiments, the straps can be repositioned to extend behind a user's ear(s). In various embodiments, the straps can be made from a variety of elastomeric materials, e.g. neoprene.

In various embodiments, a plurality of sensors can be included to measure patient vital parameters. In the exemplary embodiment shown, the sensors include a temperature sensor 140, a pulse oximeter 150, and/or a breathe sensor 160 (e.g., microphone). In various embodiments, the temperature sensor 150 can be positioned proximate the user's ear canal, and measure patient body temperature via infrared waves. In various embodiments, the pulse oximeter 150 can be positioned below the temperature sensor 140, e.g., under/behind the earlobe and proximate the jaw of the user. In various embodiments, the temperature sensor 140 and pulse oximeter 150 are disposed on an earpiece 145, e.g., C-shaped elastic clip which is biased to press the sensor(s) into engagement with the user's skin. In various embodiments, the earpiece 145 can be a discrete component that is coupled to the strap 130. In various embodiments, the earpiece 145 (e.g., clip) can be formed integrally with the strap 130.

In various embodiments, as shown in FIG. 1D, the earpiece 145 may be detachable from the strap 130, with a wire for connection to a power source (e.g., battery) and wireless (e.g., Bluetooth) module 155 disposed on the mask. In various embodiments, the temperature sensor 140 and/or pulse oximeter 150 may be removably coupled to the earpiece 145 so that either sensor can be replaced if damaged. In various embodiments, the breathe sensor (e.g., microphone) 160 may be removably coupled to the mask portion.

In various embodiments, as shown in FIGS. 1E-1F, a single strap can be employed to retain the mask on the user's face. In such embodiments, the printed circuit board 120 can be housed within a compartment in the top of the (single) strap, rather than on the mask itself

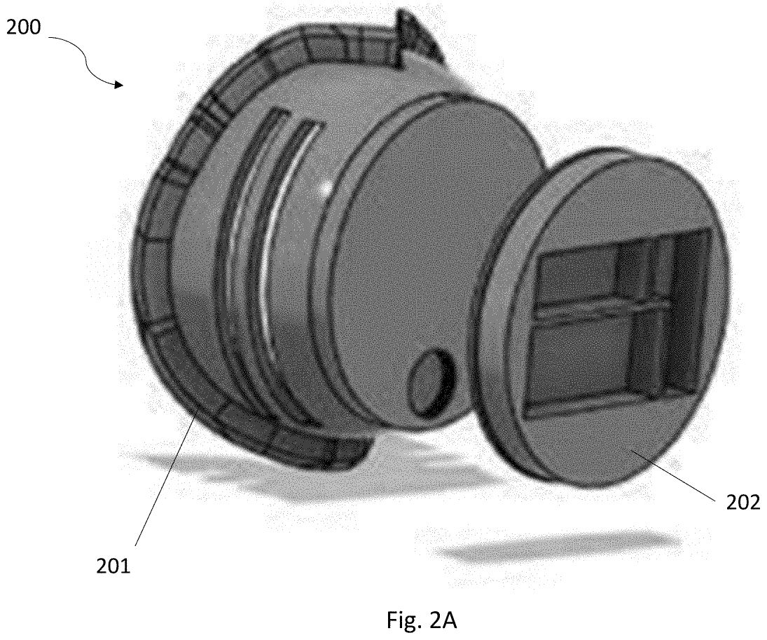

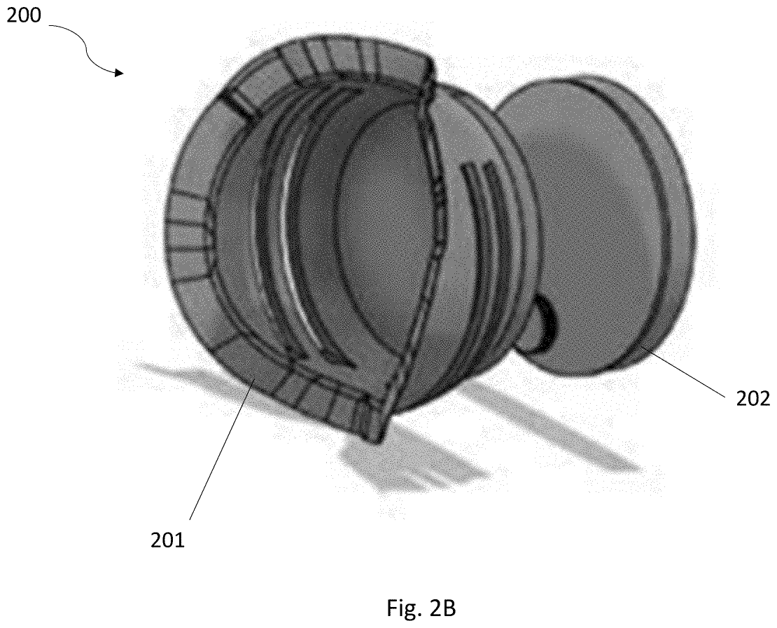

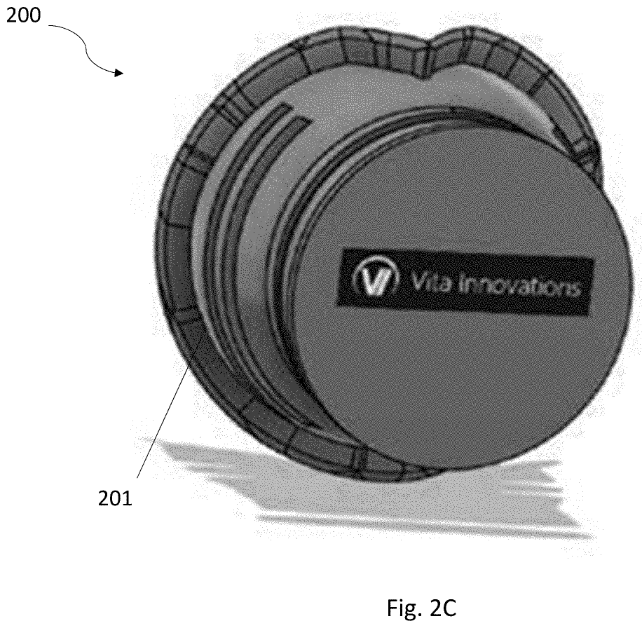

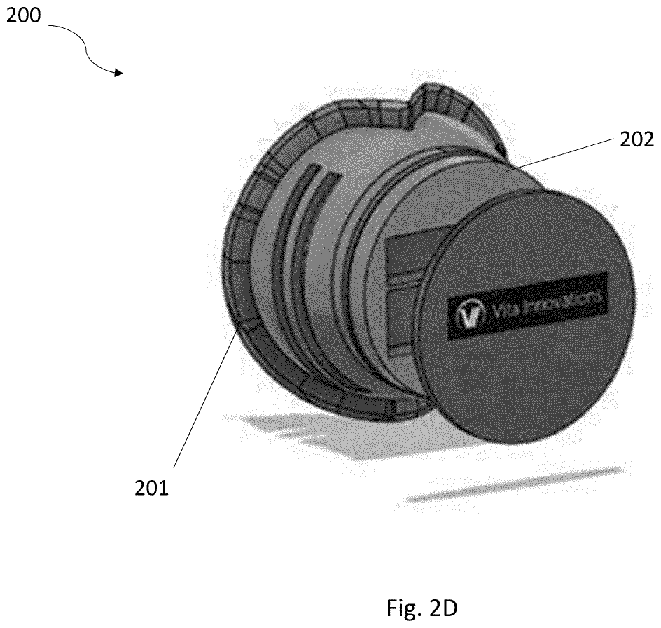

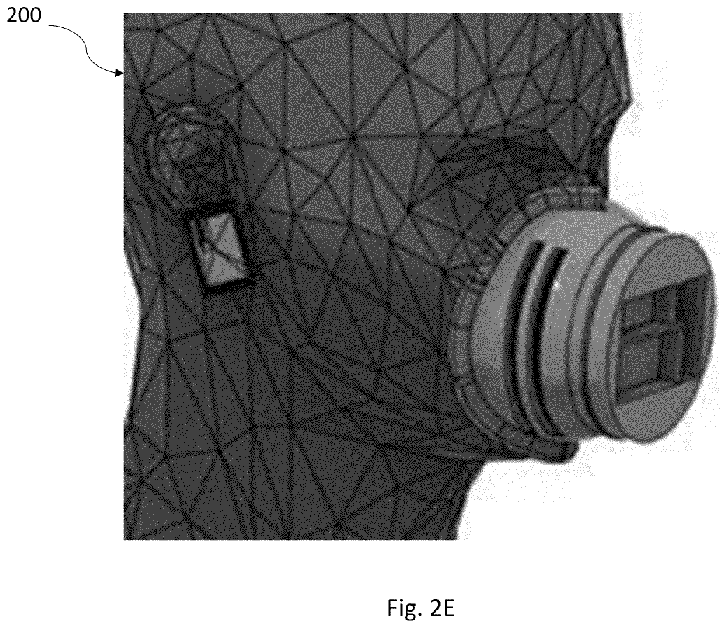

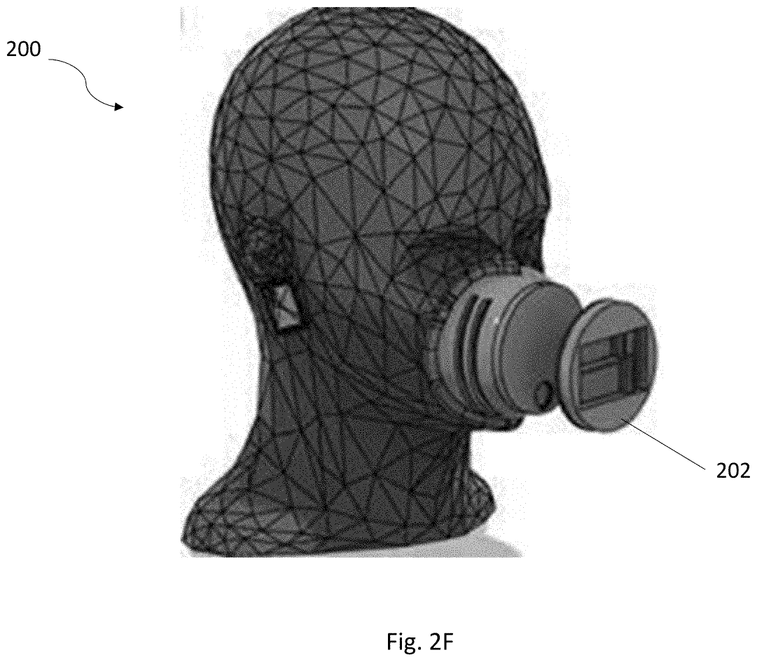

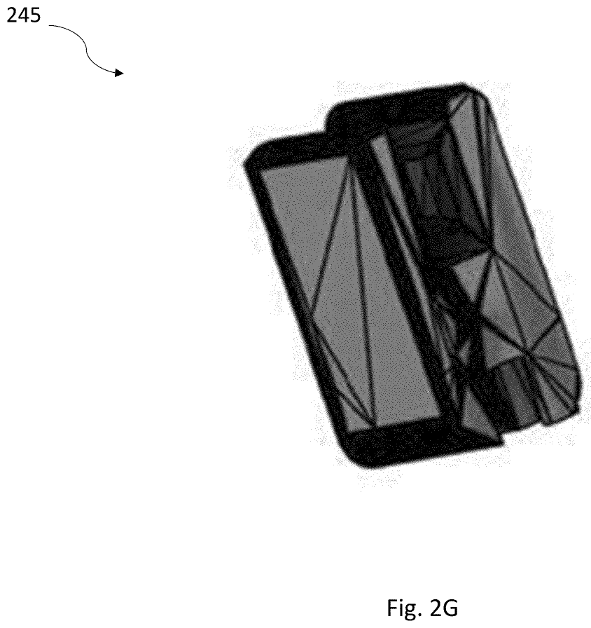

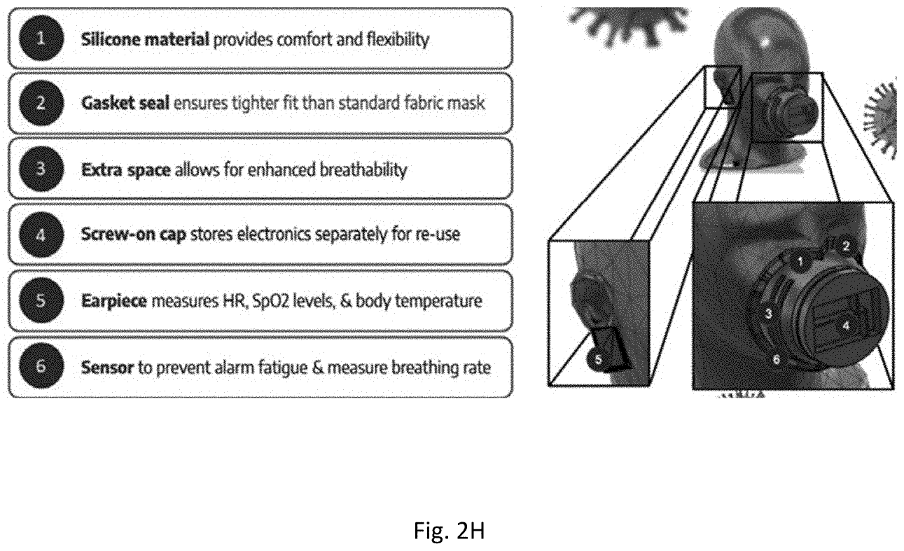

FIGS. 2A-2F illustrate an exemplary vital-monitoring mask 200, FIG. 2G illustrates an exemplary earpiece, and FIG. 2H illustrates an exemplary vital-monitoring mask 200 and earpiece 245. In various embodiments, the mask 200 includes a silicone gasket 201 and an earpiece 245. In various embodiments, the silicone gasket 201 is designed to fit underneath a standard disposable surgical/fabric mask to provide a structure that introduces greater breathability while also a more secure and comfortable fit. In various embodiments, the gasket may include an array of pressure sensors configured to contact a user's face. In various embodiments, the pressure sensors may measure breathing rate of the user via pressure measurements (e.g., electrical conductivity when pressure is applied). In various embodiments, the breathing rate measurement may be used to address hospital alarm fatigue. In various embodiments, the pressure sensors can be used to assist a healthcare professional (e.g., a nurse) in detecting if the patient has their mask on properly (or if they are risking transmission in the waiting room). In various embodiments, a notification may be generated at a workstation to bring attention to improper mask use (e.g., a mask that is not properly sealed against a user's face) via a desktop application.

As shown in FIGS. 2A-2F, the mask 200 may include an electronics compartment 202 configured to screw onto the mask 200 such that the compartment may be easily detached for sanitization and/or re-usability. In various embodiments, the gasket 201 may be affixed to a harder plastic housing. In various embodiments, the compartment may be rotatably secured on the harder plastic housing. In various embodiments, the gasket 201 may include one or more holes. In various embodiments, extra space created by the housing and/or holes in the gasket may provide for greater breathability. In various embodiments, these components, together, can be secured onto the face and held up by a standard surgical mask.

In various embodiments, the mask 200 may include a separate compartment that houses the integrated electronics (e.g., PCB, microcontroller, battery, and/or Bluetooth module) that will be in electrically communication with the external sensing device 45 (e.g., earpiece). In various embodiments, the compartment may be integral with the mask 200. In various embodiments, the compartment may be detachable so that it can be sanitized separately from the gasket and reused with new masks and/or silicone gaskets. In various embodiments, the external sensing device 45 includes a temperature sensor (e.g., infrared sensor). In various embodiments, the temperature sensor may rest inside a patient's ear to measure body temperature. In various embodiments, a portion of the earpiece (e.g., the bottom) includes a pulse oximeter which may be fastened on either side of the patient's earlobe to measure blood oxygen (SPO₂) levels and/or heart rate over time (e.g., continuously or periodically). In various embodiments, data from the breathing rate, body temperature, blood oxygen saturation levels, and/or heart rate may be recorded at the processor embedded in the central PCB. In various embodiments, the data may be stored in a computer readable storage medium. In various embodiments, the data may be transmitted via a wireless protocol (e.g., Bluetooth) to an external device (e.g., desktop monitor) to display the data.

In various embodiments, the mask may include any other suitable additional sensors and/or mechanical components as is known in the art. In various embodiments, a capnography sensor may be attached in the electronics case to measure end tidal CO₂. In various embodiments, the mask may include one or more holes in the gasket to allow for nasal cannula inserts to be placed in the mask and thereby accommodate people with breathing issues.

In various embodiments, a computer and/or mobile application may be provided for use with the vital-monitoring masks described herein. In various embodiments, the application may receive one or more of the breathing rate data, body temperature data, blood oxygen saturation data, and/or heart rate data. In various embodiments, the application may perform analytics on the received data to thereby detect any abnormalities in the data. In various embodiments, the application may include a learning system. In various embodiments, the learning system may be pre-trained. In various embodiments, the learning system may be trained (and/or retrained) on ground-truth data. For example the learning system may be trained on a combination of normal physiological data (heart rate, breathing rate, SPO₂, and/or temperature) that has been labelled as normal and abnormal physiological data (heart rate, breathing rate, SPO₂, and/or temperature) that has been labelled as abnormal.

In some embodiments, a feature vector is provided to a learning system. Based on the input features, the learning system generates one or more outputs. In some embodiments, the output of the learning system is a feature vector.

In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other trainable classifiers are suitable for use according to the present disclosure, including random decision forests, linear classifiers, support vector machines (SVM), or artificial neural networks (ANN) such as recurrent neural networks (RNN) or convolutional neural network (CNN). In various embodiments, the learning systems described herein use artificial neural networks, and more particularly convolutional neural networks.

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

In various embodiments, the application may provide a notification to a healthcare provider (e.g., a nurse) when one or more of a patient's vitals are abnormal (e.g., exceed a predetermined threshold). In various embodiments, the notification may be a push notification to a mobile device (e.g., phone or smart device). In various embodiments, the notification may be a vibration. In various embodiments, the application may provide a notification to the wearer of the mask. In various embodiments, the application may provide a notification to and/or automatically contact one or more medical specialists when certain vitals are determined to be abnormal (e.g., exceed a predetermined threshold). In various embodiments, the application may connect to two or more masks at a single computer and/or displaying the recorded vitals for each mask. In various embodiments, a user of the application may change alert thresholds for each mask. In various embodiments, the user of the application may connect a patient's mask to the computer via a pairing protocol as is known in the art. In various embodiments, the user of the application may select a specific mask to light up/create sound if thresholds exceeded (if the mask provides light and/or sound capability). In various embodiments, the application may integrate with an EMR system. In various embodiments, the application may record the patient's vitals into the patient's EMR file. In various embodiments, the user of the application may adjust (e.g., raise, lower) ventilator power according to whether a patient's (wearing the vital-monitoring mask) blood oxygen is too low or high. In various embodiments, when alerted that a patient's temperature is too high, a health care professional may apply a cold compress.

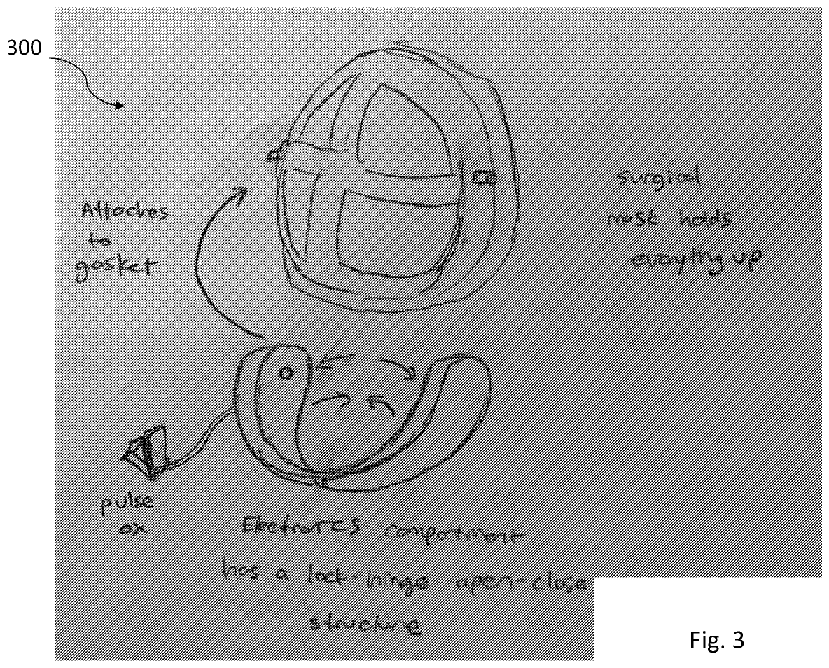

FIG. 3 illustrates an exemplary vital-monitoring mask 300 including a silicone frame is configured to create extra breathing space. In various embodiments, the silicone frame includes notches to hold onto the electronics compartment. In various embodiments, the electronics compartment is made of plastic and houses a microcontroller, PCB, battery, and Bluetooth module. In various embodiments, the case may be configured to snap onto the notches of the silicone frame to thereby hold the case in place. In various embodiments, a pulse oximeter is attached to the electronics via an electrical wire. In various embodiments, the silicone frame and the electronics compartment, once attached together, can be secured onto the face with a surgical mask.

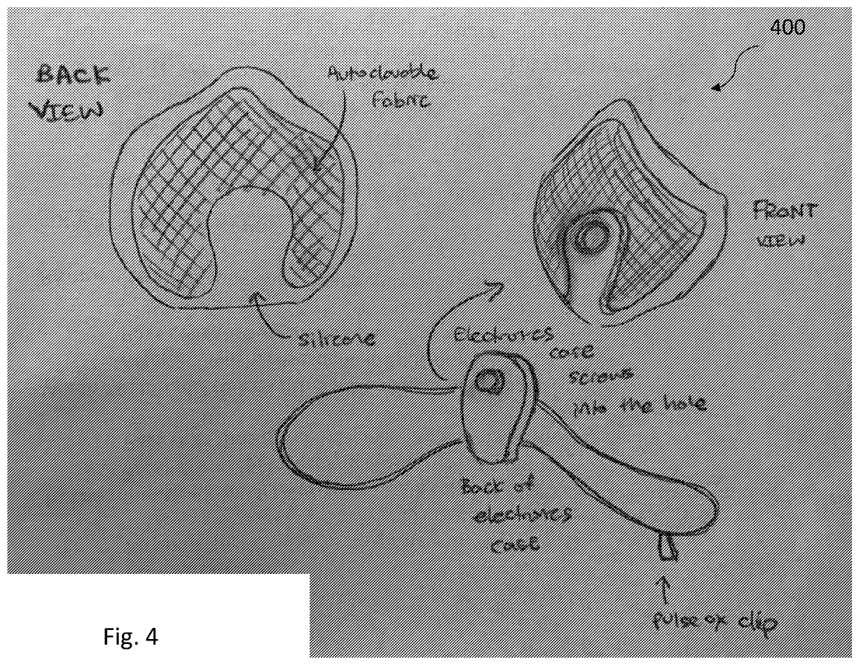

FIG. 4 illustrates an exemplary vital-monitoring mask 400 including a reusable silicone mask with a portion made of fabric to filter out particles (e.g., at the same efficiency as a surgical mask). In various embodiments, the fabric may be reusable. In various embodiments, the fabric may be autoclaved. In various embodiments, similar to the above embodiments, a plastic electronics case can be secured to the front of the mask. In various embodiments, one or more bands may secure the mask onto the face. In various embodiments, one band (e.g., the right band) has a wire within it that attaches to the ear pulse oximeter. In various embodiments, the silicone/fabric mask can be autoclaved. In various embodiments, the electronics compartment may be sanitized with vaporized hydrogen peroxide or antiseptic wipe.

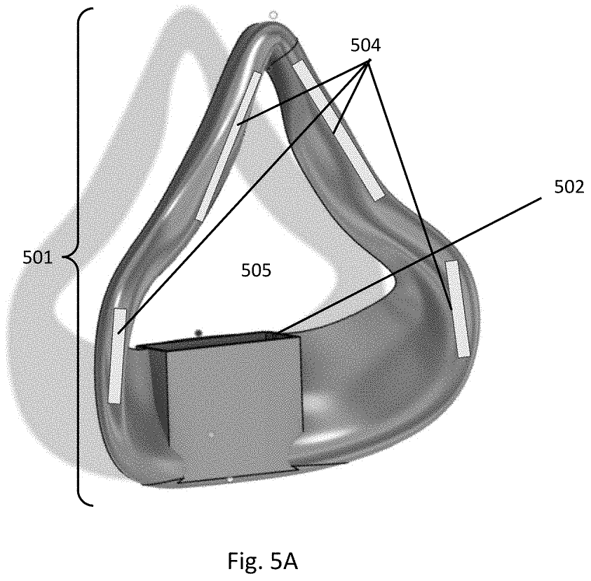

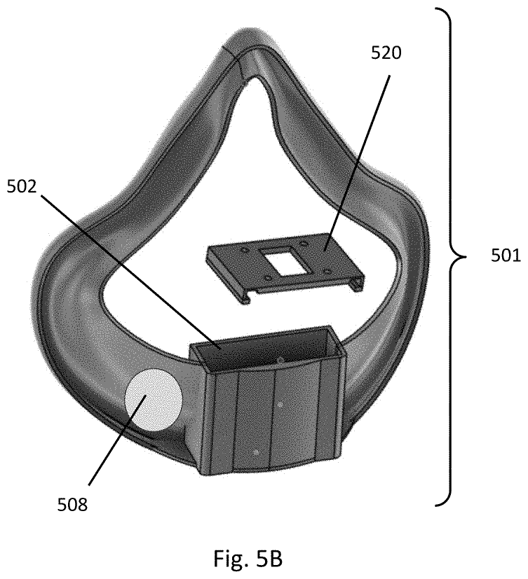

FIGS. 5A-5B illustrate a back view (FIG. 5A) and a front view (FIG. 5B) of the vital-measuring gasket mask with a built-in enclosure for insertion of the electronics. In various embodiments, the gasket 501 may be shaped to tightly conform to the shape of the patient's face. In various embodiments, gasket 101 may be attached to a patient's face to detect or measure vital signs. In various embodiments, gasket 501 may be formed from any suitable deformable and/or cushioned material. For example, gasket 501 may be formed from polymers such as silicone. In various embodiments, gasket 501 may contact and/or be pressed against a patient's face. In various embodiments, the gasket 501 is designed to conform to the shape of the face and provide a tighter fit, e.g., similar to a standard p100 mask.

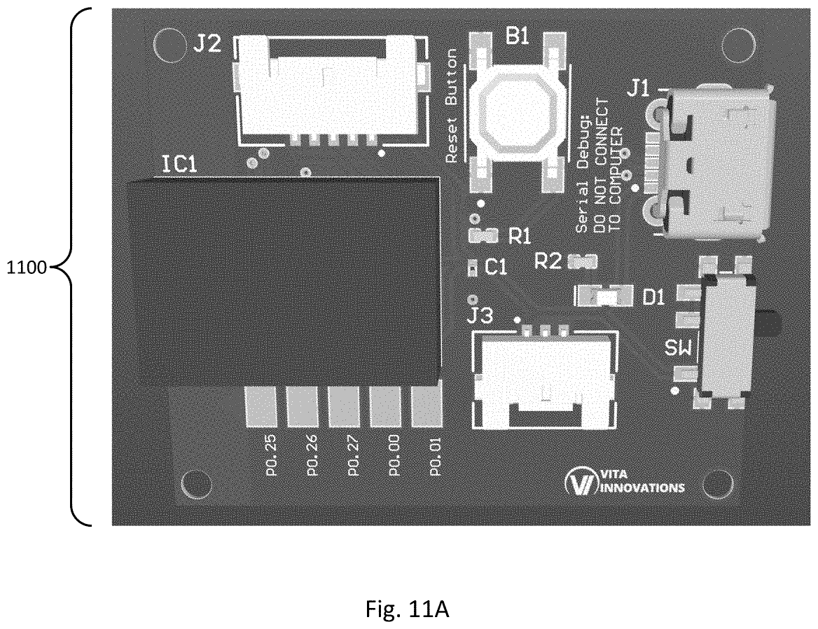

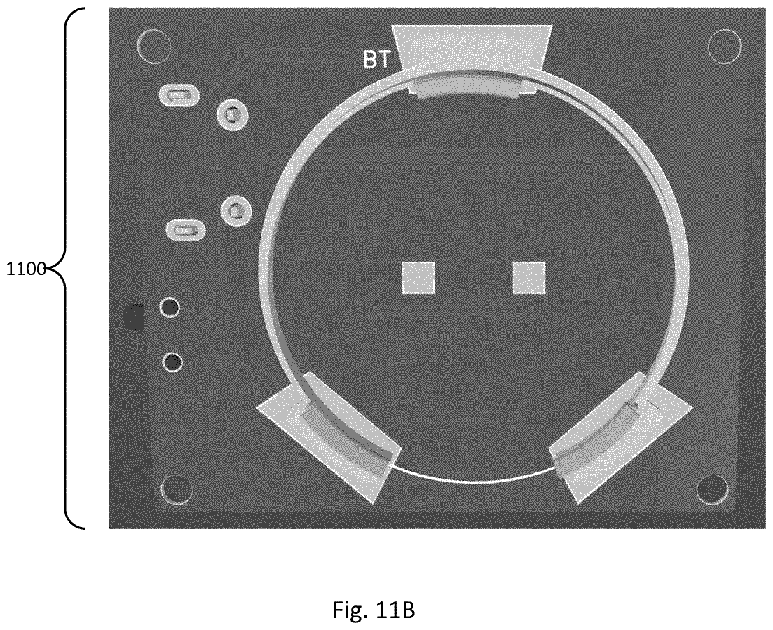

In various embodiments, gasket 501 may include an electronics housing 502. In various embodiments, the electronics housing 502 may be integral with the gasket 501. As shown in FIGS. 5A-5B, gasket 501 may be coupled, attached, and/or fixed to electronics housing 502 to provide support to electronics system 1100 shown in FIGS. 11A-11B. For example, electronics housing 502 can be formed from polymers, such as silicone, plastics, etc.

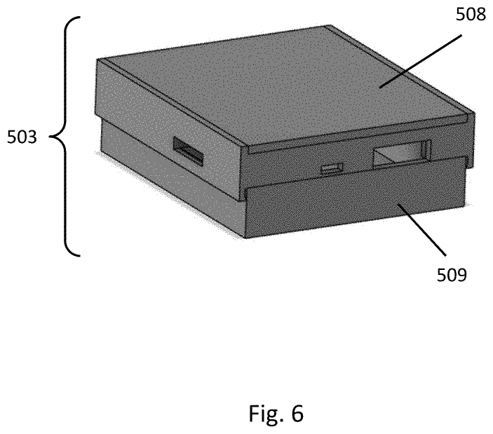

FIG. 6 illustrates a protective electronics casing 503 that houses the electronic components, allowing for the electronics to be separated and removed from the gasket for reusability and sanitization. In various embodiments, the electronics casing 503 consists of a cover and the main body that holds the electronics. In various embodiments, electronics housing 502 may hold electronics casing 503, which encases electronics system 1100. In various embodiments, electronics casing 503 may be made of a more rigid material, like plastics similar to PLA or PTEG. In various embodiments, the electronics casing 503 may include a case cover 508 and base 509. In various embodiments, gasket 501 may include a cover piece 520 that may cover and/or seal electronics housing 502 and contain or hold the electronic devices included therein.

In various embodiments, the electronics system 1100 (shown in FIGS. 11A-11B) can be contained in electronics case 503 and easily inserted into electronics housing 102 of gasket 501 and also removed for sanitization and re-usability. In various embodiments, these components, together, can be secured onto the face and held up by a fabric mask or standard surgical mask. In various embodiments, strings or straps may be attached to the sides of gasket 501 to wear on the ears and additional support. In various embodiments, the extra space created between the patient's face and fabric/surgical mask by gasket 501 provides for greater breathability compared to that of the fabric/surgical mask alone.

FIG. 5A shows the back view of gasket 501 where one or more pressure sensors 504 may be positioned. In various embodiments, the one or more pressure sensors 504 may be positioned on gasket 501 in such a way to detect when gasket 501 has been removed from, and is no longer in contact with, the patient's face. In various embodiments, opening 505 may be formed into gasket 501 to serve as a passage for air or insertion of nasal cannulas.

In various embodiments, a pulse oximeter disposed in recess 506 and electronics within the gasket 501 (e.g., electronics received by electronics housing 502) may be in communication with computing system 1000. In various embodiments, computing system 1000 is capable of obtaining measured data relating to vitals and analyzing the data.

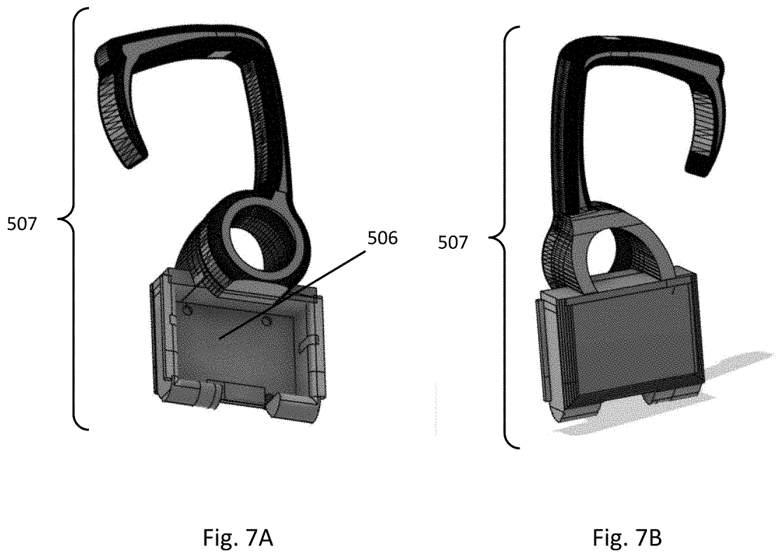

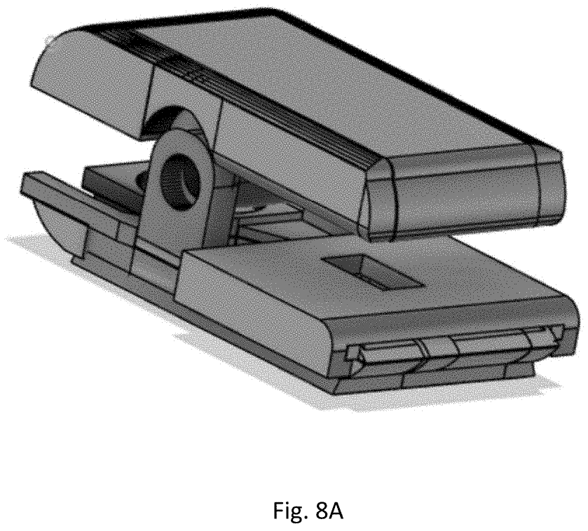

FIGS. 7A-7B illustrate a front view (FIG. 7A) and a back view (FIG. 7B) of a pulse oximeter earpiece component, which can house the pulse oximeter used to measure vitals such as blood oxygen level, heart rate, breathing rate, or otherwise. In various embodiments, the earpiece model may include an over-the-ear configuration. FIGS. 7A-7B show the pulse oximeter recess 506, located in earpiece casing 507, which stores a pulse oximeter sensor capable of measuring vitals such as blood oxygen levels, heart rate, and breathing rate. In various embodiments, the casing may be made of a rigid yet comfortable material capable of clipping onto the earlobe or attaching over the ear. In various embodiments, the earpiece casing 507 may be connected to the gasket 501 via a retractable reel 508 that functions as a retractable cord to extend from the gasket 501 to a patient's ear when in use and being worn.

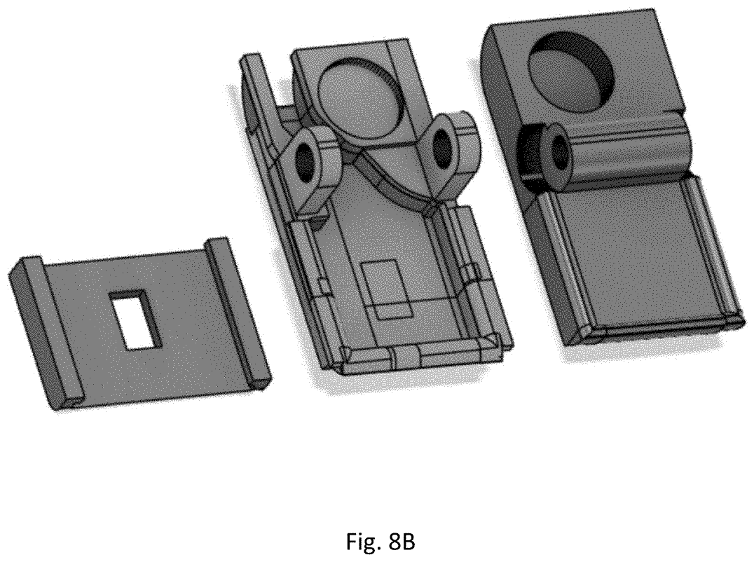

FIG. 8A illustrates an earpiece clamp that may house the pulse oximeter for clamping on the earlobe. In various embodiments, the earpiece clamp shown in FIGS. 8A-8B may be an alternative to the over-the-ear earpiece configuration as seen in FIGS. 7A-7B. FIG. 8B illustrates an exploded view of the earpiece clamp, displaying the top and bottom components as well as the intermediate cover that can be placed over the pulse oximeter.

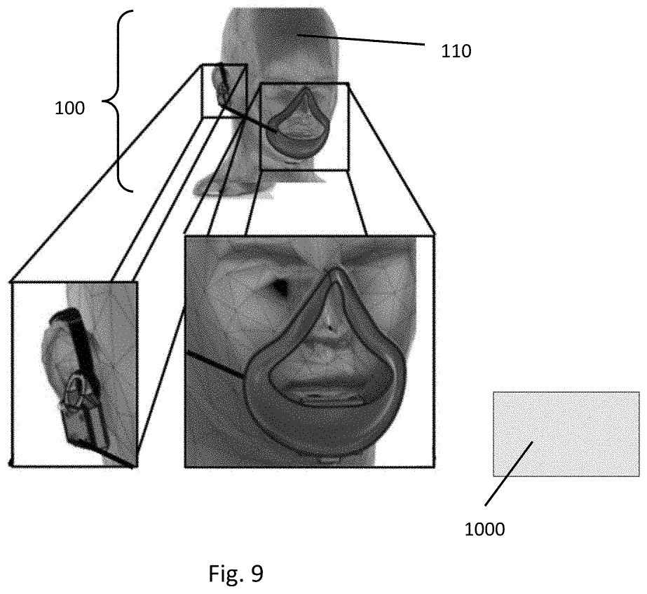

FIG. 9 illustrates a patient wearing the mask, with zoomed in windows of the pulse oximeter attached to the earlobe, connected via a retractable reel to the gasket mask on the face. In various embodiments, the apparatus is communicating with the computing system 1000 to relay information, and can be supported and held up in place by any fabric/surgical mask positioned over the gasket. FIG. 9 shows the mask 100 as an integrated system on a patient's face 10. In various embodiments, a pulse oximeter 106, which may or may not be connected to the electronics housing 102, communicates wirelessly with the computing system 1000 using a wireless protocol, such as Bluetooth. In various embodiments, other sensors, such as the pressure sensor 104 configured to measure breathing rate, or the temperature sensor configured to measure body temperature, also communicates using a wireless protocol.

In various embodiments, additional sensors and/or mechanical components suitable for determining vitals as are known in the art may be included in a mask. In one example, a capnography sensor may be attached in the electronics case to measure end tidal CO2. In another example, the mask may include holes in the silicone seal to allow for nasal cannula inserts to be placed in the mask to thereby help accommodate people with breathing issues.

In various embodiments, a silicone frame of the mask may create extra breathing space. In various embodiments, the silicone frame may include notches to hold onto the electronics compartment. In various embodiments, the electronics compartment may be made of plastic. In various embodiments, the electronics compartment may house the microcontroller, PCB, battery, and Bluetooth module. In various embodiments, the case may have the capability to snap onto the silicone frame's notches to hold it in place. In various embodiments, the pulse oximeter may be attached to the electronics by an electrical wire. In various embodiments, the silicone frame and the electronics compartment, once attached together, can be secured onto the face with a surgical mask.

In various embodiments, a mask may include a camera (e.g., digital camera) on the inside of the mask. In various embodiments, a processor may be configured to detect face drooping from camera images of the patient's face and send alerts on the desktop application signaling medical concerns, for example, signs of a stroke. In various embodiments, the mask may include one or more electromyography (EMG) sensors on the face of the user. In various embodiments, the EMG sensor may be positioned next to the pressure sensors as another way to confirm the presence of a stroke.

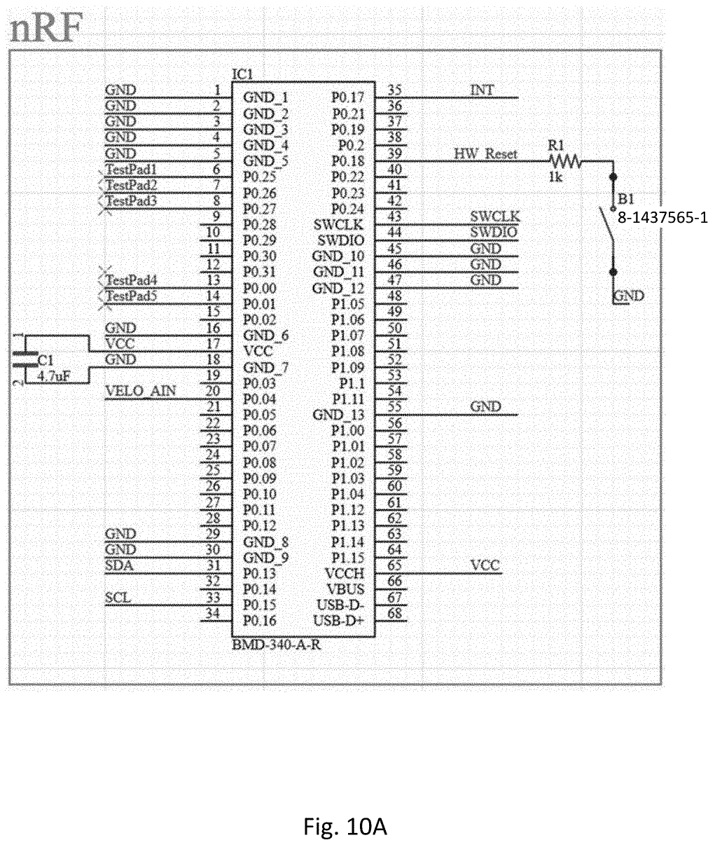

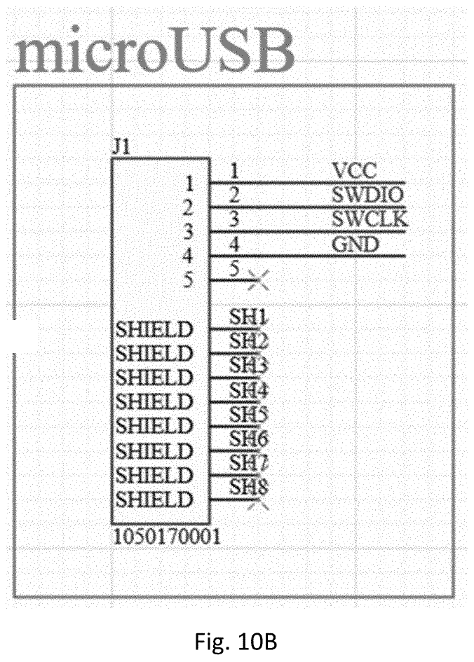

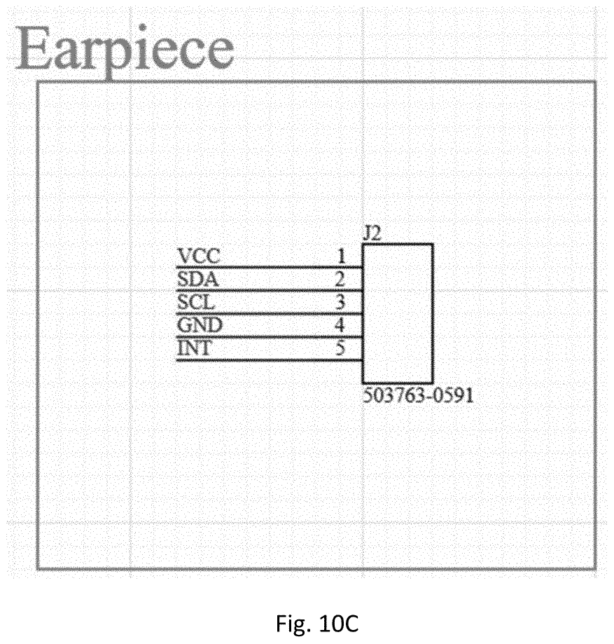

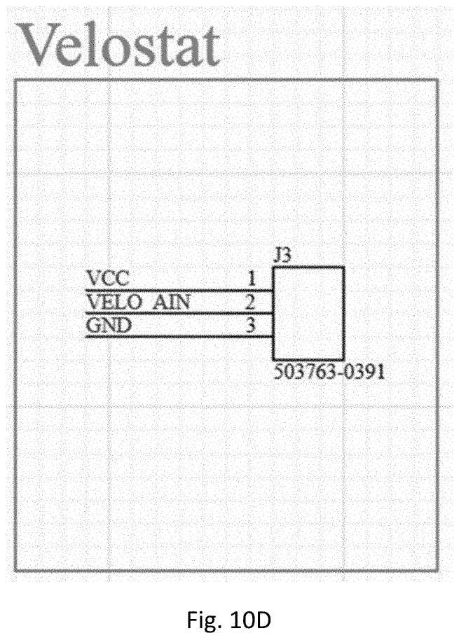

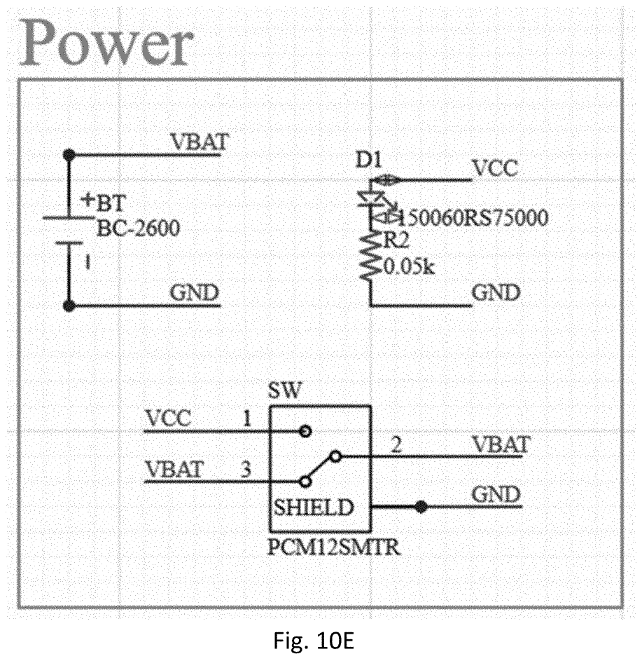

FIGS. 10A-10E illustrate schematics of the VitalMask PCB. In various embodiments, the earpiece module depicted are wires that go directly to the MAX30102 pulse oximeter sensor and the MLX90614 temperature sensor. In various embodiments, the Velostat module depicted are wires that go directly to our custom made velostat pressure sensor made from velostat (sandwiched) between two layers of a conductive sheet (in the case conductive fabric). In various embodiments, the Power module depicted shows VBAT and GND which are wires that go directly to the battery on the PCB.

FIGS. 11A-11B illustrate a 3D model of the PCB with its front containing the microcontroller, support circuitry and connectors to the sensors, and the back being used as the battery holder to power the system.

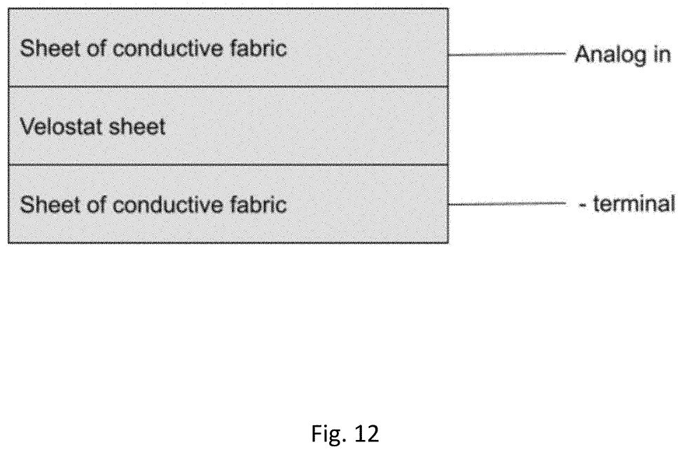

FIG. 12 illustrates a velostat sensor design. In various embodiments, the “analog in” terminal connects to the VELO AIN wire on the PCB, while the negative terminal connects to ground with a series resistor. In various embodiments, the pressure sensor acts as a variable resistor to change voltage when pressure is applied.

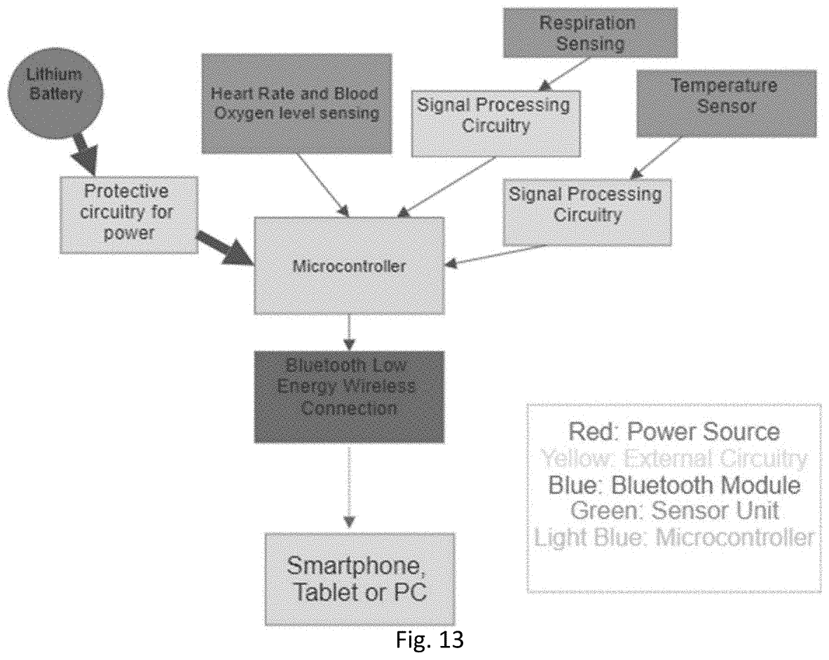

FIG. 13 illustrates a schematic representation of an electrical system of a wearable biosensor mask in accordance with an embodiment of the present disclosure. As shown in FIG. 13, the data collected from the IoT sensors incorporated into the apparatus may be wirelessly transmitted, e.g., via Bluetooth, from an on-board microcontroller to a desktop (e.g., PC) or mobile (e.g., tablet) application that visualizes and analyzes the information in real time. In various embodiments, the mobile application may be programmed in Swift, React, Kotlin, and/or Java. In various embodiments, the desktop application may be programmed in Java, Python, and/or C++. Furthermore, the data can be integrated into patient records and be stored on a cloud-based system for further access and analysis.

In some embodiments, the sensors can alert the user and/or medical staff if an errant signal is detected (e.g., lack of measurement, or measurements outside acceptable pre-programed limits) which can reveal a defective seal/placement of the mask on the user. For example, if the temperature and blood oximeter sensors are able to monitor their respective vital signs, but the breathe sensor does not detect any readings, an alert can be generated to adjust the mask and/or replace the sensor.

Additionally, in some embodiments, memory can be incorporated into the device so as to store the vital measurements monitored during use. This can be advantageous in the event that transmission to the health care facility is interrupted for an extended period of time, preventing data loss. Transmission of the data stored on the mask can resume once connection is reestablished.

Thus, the present disclosure provides a mask which is reusable, has a removable filter, has companion applications, and monitors certain vitals. Although the exemplary embodiments disclosed herein depict particular locations for the various sensors incorporated, it should be understood by artisans of ordinary skill that the location, number and size of the sensor(s) can be varied as so desired to accommodate masks of different sizes, shapes and material construction.

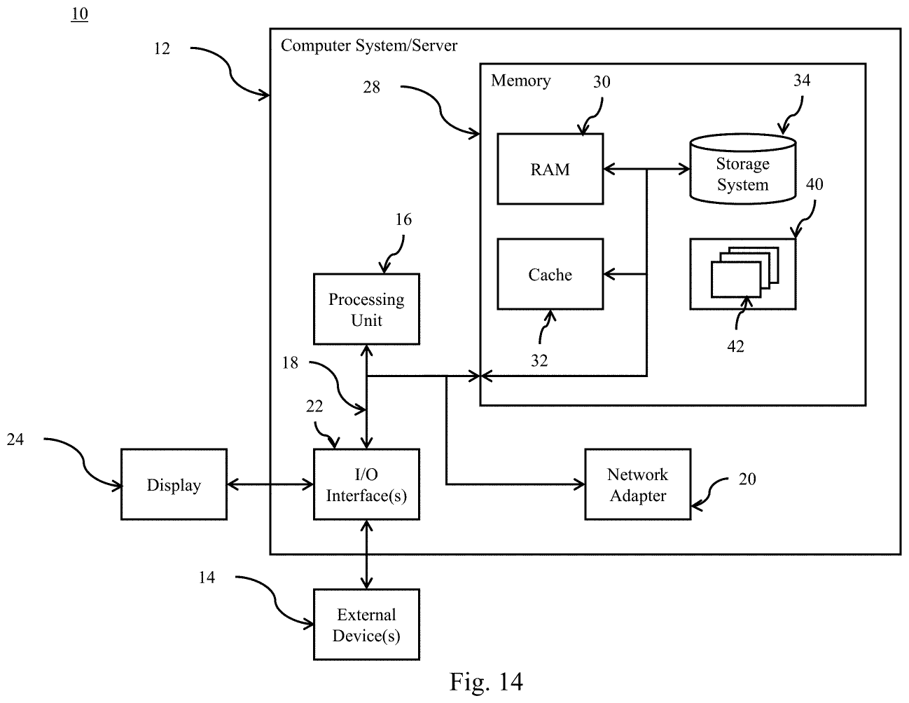

Referring now to FIG. 14, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 14, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

1. An apparatus comprising:
a respiratory mask having a gasket configured to form a seal against skin around a nose and a mouth of a user, the mask having a breath sensor, at least one opening, and a filter disposed in the at least one opening;
a sensing device comprising a temperature sensor, a blood oxygen saturation sensor, and a heart rate sensor, the sensing device configured to attach to an external body part of the user and externally from the mask; and
an electronics housing disposed on the mask, the electronics housing comprising a power source and a processor in electrical communication with the breath sensor, the temperature sensor, the blood oxygen saturation sensor, and the heart rate sensor,
wherein the electronics housing comprises a computer readable storage medium having program instructions embodied therewith, the program instructions executable by the processor to cause the processor to perform a method comprising:
receiving breathing rate data from the breath sensor;
receiving temperature data from the temperature sensor;
receiving blood oxygen saturation data from the blood oxygen saturation sensor;
receiving heart rate data from the heart rate sensor; and
transmitting the breathing rate data, temperature data, blood oxygen saturation data, and heart rate data to an external device.

2. The apparatus of claim 1, wherein the mask further comprises one or more electromyography (EMG) sensors configured to contact the skin and in electrical communication with the processor.

3. The apparatus of claim 2, wherein the processor is further configured to receive EMG data from the one or more EMG sensors.

4. The apparatus of claim 1, wherein the mask further comprises a camera disposed within the mask and configured to image at least a portion of a face of the user.

5. The apparatus of claim 4, wherein the processor is further configured to receive image data from the camera.

6. The apparatus of claim 1, wherein the mask further comprises one or more pressure sensors configured to detect whether the mask substantially seals the skin around the nose and mouth of the user.

7. The apparatus of claim 1, wherein the sensing device is configured to removably attach to an ear of the user.

8. The apparatus of claim 1, wherein the breath sensor comprises a microphone.

9. The apparatus of claim 1, wherein the breath sensor comprises a pressure-sensitive conductive fabric.

10. The apparatus of claim 1, wherein the power source comprises one or more batteries.

11. The apparatus of claim 1, wherein the gasket comprises silicone.

12. (canceled)

13. (canceled)

14. A system comprising:
the apparatus of claim 1; and
the external device.

15. The system of claim 14, wherein the external device comprises a mobile device.

16. (canceled)

17. The system of claim 14, wherein the external device comprises a remote server.

18. (canceled)

19. The system of claim 14, wherein the external device comprises a computer readable storage medium having program instructions embodied therewith, the program instructions executable by the processor to cause the processor to perform a method comprising:
receiving breathing rate data, temperature data, blood oxygen saturation data, and heart rate data from the apparatus;
determining an abnormality in at least one of the breathing rate data, temperature data, blood oxygen saturation data, and heart rate data;
when an abnormality is detected, providing a notification to a healthcare provider.

20. The system of claim 19, wherein determining an abnormality comprises:
determining a feature vector from at least one of the breathing rate data, temperature data, blood oxygen saturation data, and heart rate data; and
determining, at a trained learning system, the abnormality based on the feature vector.

21. The system of claim 19, wherein the method further comprises training the learning system on the breathing rate data, temperature data, blood oxygen saturation data, and heart rate data from the apparatus.

22. A vital-monitoring apparatus comprising:
a sensing device comprising a temperature sensor, a blood oxygen saturation sensor, and a heart rate sensor, the sensing device configured to attach to an external body part of a user; and
an electronics housing configured to be removably attached to a respiratory mask, the electronics housing comprising a power source and a processor in electrical communication with the breath sensor, the temperature sensor, the blood oxygen saturation sensor, and the heart rate sensor,
wherein the electronics housing comprises a computer readable storage medium having program instructions embodied therewith, the program instructions executable by the processor to cause the processor to perform a method comprising:
receiving breathing rate data from the breath sensor;
receiving temperature data from the temperature sensor;
receiving blood oxygen saturation data from the blood oxygen saturation sensor;
receiving heart rate data from the heart rate sensor; and
transmitting the breathing rate data, temperature data, blood oxygen saturation data, and heart rate data to an external device.

23. (canceled)

24. (canceled)

25. (canceled)

26. (canceled)

27. A kit comprising:
the apparatus of claim 22;
the respiratory mask onto which the electronics housing is configured to removably attach.

28. (canceled)

29. (canceled)

30. (canceled)

31. (canceled)

32. A vital-monitoring device comprising:
a sensing device comprising a temperature sensor, a blood oxygen saturation sensor, and a heart rate sensor, the sensing device configured to attach to an external body part of a user.

33. (canceled)

34. (canceled)

35. (canceled)

36. (canceled)
